(19) United States
(12) Patent Application Publication
(10) Pub. No.: US 2006/0060199 A1
(43) Pub. Date: Mar. 23, 2006

(54) Self-inflating resuscitation system

(75) Inventors: Samsun Lampotang, Gainesville, FL (US); Nikolaus Gravenstein, Gainesville, FL (US)

Correspondence Address: AKERMAN SENTERFITT, P.O. BOX 3188, WEST PALM BEACH, FL, 33402-3188, US

(73) Assignee: University of Florida, Gainesville, FL (US)

(21) Appl. No.: 11/234,839

(22) Filed: Sep. 23, 2005

Related U.S. Application Data
(60) Provisional application No. 60/612,386, filed on Sep. 23, 2004.

(51) Int. Cl. A61M 16/00

(52) U.S. Cl. 128/205.13; 128/202.28

(57) ABSTRACT

A self-inflating resuscitation system formed from a self-inflating resuscitation bag and an exhalation indicator. In particular, the self-inflating resuscitation bag provides an exhalation indicator, which may be an audio or visual indicator, or both. The exhalation indicator enables a caregiver to more accurately determine whether a patient is being ventilated, unlike prior art self-inflating resuscitation bags, and helps to detect esophageal intubation in intubated patients or gastric trapping of gas in non-intubated patients undergoing positive pressure ventilation. In general, the device may be interposed between any source of positive pressure ventilation and any airway device to monitor exhalation as an indicator of adequacy of ventilation.

### CROSS-REFERENCE TO RELATED APPLICATIONS

This application claims the benefit of U.S. Provisional Patent Application No. 60/612,386, filed Sep. 23, 2004.

### FIELD OF THE INVENTION

The present invention is directed to an article of manufacture useful during positive pressure ventilation and particularly as a self-inflating resuscitation bag (SIRB), commonly known as an Ambu bag. In particular, the present invention is a self-inflating resuscitation bag or kit that better enables a caregiver to determine whether a patient is being adequately ventilated by using the principle of monitoring exhalation as an indicator of ventilation. The apparatus may also be used to detect inadequate ventilation caused among others by a poor seal (e.g., in a face mask, laryngeal mask airway, endotracheal tube cuff), airway obstruction, poor technique (e.g., too short inspiratory time or too fast respiratory rate) or a defective SIRB and, in an intubated patient, esophageal intubation. The invention may be interposed between any source of positive pressure ventilation (such as an SIRB or a ventilator) and any airway device (e.g., endotracheal tube, laryngeal mask airway, face mask) or device used to interface between the source of positive pressure ventilation and the patient. The invention may also be used to detect esophageal intubation by observing the refill of a collapsed SIRB when the entrainment port is connected to an endotracheal tube placed in a patient. The invention may also be used as an emergency suctioning device by connecting the entrainment port of a collapsed SIRB to the area to be suctioned. The device or kit may also be used as a training device that provides real-time feedback to novices, students and practitioners undertaking refresher courses. The self-inflating resuscitation bag may also be used during cardiac arrest in resuscitation efforts.

### BACKGROUND OF THE INVENTION

Resuscitation, as that term is herein used, refers generally to externally exerted efforts to assist or restore breathing of a patient whose natural breathing has either become impaired or has ceased, or to at least temporarily attempt to emulate the effects of more natural breathing in the patient. Resuscitation involves forcing air or oxygen under appropriate pressure through the patient's airway system and into his lungs to inflate the latter at appropriate intervals separated by periods during which such application of air or oxygen under pressure is interrupted to permit the previously applied gas to escape from the patient's lungs and the latter to deflate. Effective resuscitation requires adequate ventilation. It is common for mask ventilation to be compromised by substantial leakage of the gas intended for ventilation from, for example, around an imperfect face seal at an interface between a facemask and a patient's face. Facemask ventilation using high inflation pressures, short inspiratory times and esophageal intubation are also a cause of inadequate ventilation because gas is directed to the stomach instead of the lungs. Airway obstruction or a defective SIRB (e.g., missing a valve leaflet) will also result in ineffective ventilation that may not be evident when attempting ventilation with an SIRB.

Resuscitation bag valve mask (BVM) assemblies are commonly used in emergency care and critical care situations. When used in the field, BVMs deliver gas under positive pressure to a patient not capable of adequately breathing independently.

Manual resuscitators using self-inflating bags are well recognized in the prior art. Such devices are often used during cardio-pulmonary resuscitation (CPR) and advanced cardiac life support (ACLS). Such procedures require that a patient be supplied with large quantities of air or oxygen. In addition to forcing a volume of gas to the patient, such devices must also allow for a patient to inhale or exhale under his or her own ability. As a result, resuscitation bags are usually comprised of three basic components; to wit: a mask, a specific directional control valve arrangement, and a squeezable bag, which is typically self-inflating in the sense that the bag springs back to its molded full shape after being squeezed.

A facemask cushion is used to form a seal about the patient's nose and mouth. The facemask is typically made of a soft, pliable material and is sufficiently flexible so as to contour fit to a wide variety of facial features. Typically, a body of the mask must be sufficiently rigid to allow uniform force to be applied so as to make an airtight seal.

A directional control valve located adjacent the mask allows air or oxygen to be forced under pressure to a patient and permits the patient to exhale. In addition, the valve typically allows the patient to breathe spontaneously by drawing air through the bag and to exhale as well. Air is not forced under pressure to the patient during spontaneous breathing.

Conventional resuscitator bags, commonly called “squeeze bag” or “bag-valve-mask” resuscitators, employ some type of manually compressible and self-restoring bag having the interior thereof in fluid communication with a face mask. In its most primitive conceptual form, a resuscitator bag is used for resuscitation purposes by applying the mask to the face of a patient, manually squeezing the bag to force gas from the bag through the mask and into the patient's lungs, ceasing to squeeze the bag and removing the mask from the patient's face to permit escape of gas from the patient's lungs. Once squeezing the bag has ceased, the bag self-inflates with fresh ambient gas from atmospheric air or oxygen supplied via a one-way inlet valve from an entrainment reservoir. The entrainment reservoir is an enclosed volume open to the atmosphere where gases collects during exhalation and is drawn from during inspiration via the one-way inlet valve. The bag remains in a restored condition until the next bag squeezing operation, at which time, the cycle is repeated. A squeeze bag resuscitator thus permits a trained person administering treatment to directly control both the quantity and quality, such as with supplemental O₂, of gas forced into a patient's lungs and may control the intervals of administration to best suit the condition of the patient through choice of the extent and timing of squeezing of the bag.

Even relatively early squeeze bag resuscitators soon incorporated various refinements, including employment of resilient squeeze bags adapted to be conveniently held in one hand with the face masks carried more or less directly on the frontal extremities of the bags to increase portability and facilitate use by a single person. A bag fill valve such as an inward flow permitting check valve creating a pathway between the interior of the bag and the atmosphere or an entrainment reservoir was introduced to permit refilling of the bag with fresh air or oxygen, or both, during the restoration (exhalation) phase without removing the mask from the face of the patient. And, in conjunction with the bag fill valve, the patient non-rebreathing valve assembly was added to the squeeze bag resuscitator. Such assembly is interposed between the bag and the mask and permits fresh air or oxygen to move from the bag into the mask during the squeeze phase or during spontaneous inspiration. The non-rebreathing valve assembly vents gas returned to the mask from the patient's lungs to the atmosphere during the bag restoration or exhalation phases, thereby preventing passage of the expired gas into the bag from which it would be directed back into the patient's lungs or “rebreathed” during the next breathing cycle.

Nevertheless, conventional self-inflating resuscitation bags do not provide any means for enabling the caregiver to ascertain whether the patient is properly ventilated and is, therefore, actually or substantially exhaling. As self-inflating resuscitation bags re-inflate as a function of their design, they behave in undifferentiated fashion regardless of whether the SIRB-ventilated patient is actually being ventilated or not. This is important because, for example, if the seal between the apparatus with face mask and the patient's face leaks or gas is substantially going into the esophagus, or both, the user needs to know that the seal or the airway, or both, may be optimized to effect the desired ventilation.

In addition, the mental model that anesthesiologists associate with a bag that refills during exhalation is that of the flaccid breathing bag on the anesthesia machine. In the case of the anesthesia machine breathing bag, refilling is not automatic, i.e., the bag is not self-inflating and refilling of the bag during exhalation is an indicator of adequate exhalation at low fresh gas flows. Thus, anesthesiologists are particularly at risk of being misled by the re-inflation of an SIRB irrespective of whether effective ventilation is being delivered.

In situations where the SIRB is used with an intubated patient, a facemask is not used. However, problems still exist. For instance, an endotracheal tube may be inadvertently and unknowingly inserted in the esophagus. In this circumstance, the gas delivered to the patient when the bag is squeezed goes into the stomach rather than the lungs. The gastric sphincters may trap the gas in the stomach causing inadequate exhalation. In another instance, the endotracheal tube cuff may be under-inflated or may not provide an effective seal, creating a leak and compromising ventilation.

A calorimetric indicator that changes color in response to the concentration of CO₂ in the exhaled breath has also been used. A problem with colorimetric indicators is that CO₂ excretion stops during cardiac arrest. The resulting lack of color change even though the patient is being adequately ventilated may be disconcerting and misleading to first responders.

Accordingly, a need exists for a self-inflating resuscitation bag that may be used on a patient that is capable of better enabling the caregiver to determine whether the patient is exhaling, thereby enabling the caregiver to more accurately evaluate whether or not the patient is being properly ventilated.

### SUMMARY OF THE INVENTION

The invention is directed to a device and method for assisting breathing and determining whether a breathing is taking place. In particular, the invention is directed to a self-inflating resuscitation system includes a self-inflating resuscitation bag with one or more exhalation indicators for determining whether a patient is properly ventilated. The exhalation indicators may include, but are not limited to, audible indicators, visual indicators, electronic indicators or any combination thereof. The indicators may be added or retrofitted to a standard self-inflating resuscitation bag, may be formed as an integral part of new self-inflating resuscitation bags, may be stand-alone units that are attached to airway devices that are not necessarily connected to a self-inflating resuscitation bag and may be stand-alone units that are attached to ventilation devices other than self-inflating resuscitation bags. The exhalation indicators may also be integrally formed with the outlet valve assembly.

In particular, the self-inflating resuscitation system may include a self-inflating resuscitation chamber formed from at least one flexible outer wall having a resting position at which the self-inflating resuscitation chamber is expanded to form a cavity holding a gas and having sufficient structural integrity such that when a compressive force is removed from an outer surface of the outer wall, the outer wall returns to the resting position. In addition, the self-inflating resuscitation system may include at least one exhalation indicator coupled to the self-inflating resuscitation chamber to indicate whether a patient to which the resuscitation bag is attached is exhaling.

In another embodiment, the self-inflating resuscitation system may include a self-inflating resuscitation chamber formed from at least one flexible outer wall having a resting position at which the self-inflating resuscitation chamber is expanded to form a cavity holding a gas and having sufficient structural integrity such that when a compressive force is removed from an outer surface of the outer wall, the outer wall returns to the resting position, an intake port in the self-inflating resuscitation chamber, and an inlet valve coupled to the intake port allowing gases to flow into the self-inflating resuscitation chamber but restricting gases from flowing out of the self-inflating resuscitation chamber. The self-inflating resuscitation system may also include an outlet port in the self-inflating resuscitation chamber, an outlet valve assembly coupled to the outlet port allowing gases to flow out of the self-inflating resuscitation chamber but restricting gases from flowing into the self-inflating resuscitation chamber, and at least one exhalation indicator coupled to the outlet valve assembly to indicate whether a patient to which the resuscitation bag is attached is exhaling.

In yet another embodiment, the self-inflating resuscitation system includes a method of determining whether a patient is adequately ventilated, which includes attaching a self-inflating resuscitation bag to the patient, wherein the self-inflating resuscitation chamber comprises at least one flexible outer wall having a resting position at which the self-inflating resuscitation chamber is expanded to form a cavity holding a gas and having sufficient structural integrity such that when a compressive force is removed from an outer surface of the outer wall, the outer wall returns to the resting position and including at least one exhalation indicator coupled to the self-inflating resuscitation chamber to indicate whether a patient to which the resuscitation bag is attached is exhaling, applying a compressive force to the self-inflating resuscitation bag to expel the gas from the self-inflating resuscitation chamber into the patient, and monitoring the at least one exhalation indicator to determine whether the patient exhales.

In another embodiment, the self-inflating resuscitation system includes a method of determining whether a patient is adequately ventilated, which includes attaching a self-inflating resuscitation bag to the patient, wherein the self-inflating resuscitation chamber comprises at least one flexible outer wall having a resting position at which the self-inflating resuscitation chamber is expanded to form a cavity holding a gas and having sufficient structural integrity such that when a compressive force is removed from an outer surface of the outer wall, the outer wall returns to the resting position and including at least one exhalation indicator coupled to the self-inflating resuscitation chamber to indicate whether a patient to which the resuscitation bag is attached is exhaling, allowing the patient to spontaneously inhale gas in the self-inflating resuscitation bag, and monitoring the at least one exhalation indicator to determine whether the patient exhales.

In another embodiment, the self-inflating resuscitation system for resuscitation includes an outlet valve assembly adapted to be coupled to an outlet port of a self-inflating resuscitation bag and including an outlet valve adapted to allow gases to flow out of the self-inflating resuscitation bag but restricting gases from flowing into the self-inflating resuscitation bag and adapted to be coupled to an airway device extending from an airway of a patient, and at least one exhalation indicator coupled to the outlet valve assembly to indicate whether a patient to which the resuscitation bag is attached is exhaling. In at least one embodiment, the exhalation indicator may be positioned downstream from the outlet port.

An additional method of determining if an endotracheal tube is correctly placed in the trachea, instead of mistakenly in the esophagus, is to connect the exhalation indicator, either as a stand-alone device or as part of a larger system such as a self-inflating resuscitation bag, to the proximal port of the endotracheal tube. Subsequently pressing on the chest will cause gas to be expelled from the lungs and mimic an exhalation, thus triggering the exhalation indicator, if the endotracheal tube is correctly located in the trachea. If the endotracheal tube has been accidentally placed in the esophagus, gas being expelled from the lungs by pressing on the chest will not trigger the exhalation indicator. Conversely, pressing on the belly should expel some gas from the stomach and trigger the exhalation indicator if the tube is in the esophagus, thus confirming esophageal intubation. Confirmation of placement of a tube in the esophagus is desirable if the tube is a feeding tube, to avoid the catastrophic consequences of accidentally delivering nutritional fluid to the lungs.

The exhalation indicator may indicate exhalation flow in a binary format, such as no exhalation flow has occurred or exhalation flow is present, or may indicate exhalation flow in a proportional format. For example, for an audible indicator such as the reed in a harmonica, the sound produced may be louder in volume, or of a higher frequency, tone, pitch or timbre, as the exhalation flow rate increases or as the exhaled volume increases, or both. For a visual indicator, the visual signal may be modulated by either the exhalation flowrate or volume. For example, the rate of spinning of a turbine flowmeter may increase or the intensity of an LED or the frequency of a flashing LED indicating exhalation may be modulated according to the exhalation flowrate or volume, or both. Different mechanisms, such as placing a parallel manifold containing audible indicators of different tones and sensitivities at the exhalation port, may be used to achieve a signal proportional to the exhalation flowrate or volume, or both.

### BRIEF DESCRIPTION OF THE DRAWINGS

The accompanying drawings, which are incorporated in and form a part of the specification, illustrate embodiments of the presently disclosed invention and, together with the description, disclose the principles of the invention.

FIG. 1 is a perspective view a self-inflating resuscitation bag showing aspects of the present invention.

FIG. 2 is a cross-sectional view of the self-inflating resuscitation bag of FIG. 1 taken along section line 2-2 and shown in a resting position.

FIG. 3 is a cross-sectional view of the self-inflating resuscitation bag of FIG. 2 shown during inspiration.

FIG. 4 is a cross-sectional view of the self-inflating resuscitation bag of FIG. 2 shown during exhalation.

FIG. 5 is a schematic diagram of a self-inflating resuscitation system of this invention during use.

FIG. 6 is a cross-sectional view of the self-inflating resuscitation bag of FIG. 2 in which an oxygen source is attached to the inlet valve.

### DETAILED DESCRIPTION OF THE INVENTION

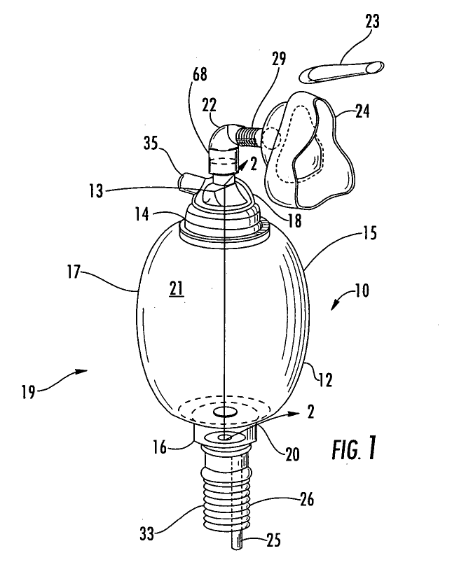

As shown in FIGS. 1-6, the present invention is directed to a self-inflating resuscitation system 10 for resuscitation of humans or animals, or both. The self-inflating resuscitation system 10 may include a self-inflating resuscitation bag 12 and one or more exhalation indicators 13 for indicating whether patients are adequately exhaling, and are therefore properly ventilated. The exhalation indicator 13 may be selected from an audible indicator, a visual indicator, an electronic indicator, or a combination thereof, such as an audible indicator and a visual indicator. The exhalation indicator 13 may be electronic or non-electronic. The audible indicator 13 may be any type of audible indicator 13 that would enable a caregiver to audibly ascertain whether a patient was exhaling. Examples of audible indicators 13 useful in the present invention include, but are not limited to, a whistle, such as, but not limited to a pea or pea-less whistle, or other appropriate whistle, a reed, a buzzer, a bell, a beeper, a ringer, a Helmholtz resonator or a combination thereof. The audible indicator 13 may be binary or proportional as previously mentioned.

The visual indicator 13 may be any type of visual indicator 13 that would enable a caregiver to visually ascertain whether a patient was exhaling. Examples of visual indicators 13 useful in the present invention include, but are not limited to, one or more lights, one or more light-emitting diodes (LEDs), a liquid crystal display (LCD), a turbine vane flow sensor used with a Ohmeda 5420 volume monitor, a pneumatic toggled lens, or any combination thereof. The turbine may include colors that change with peak expiratory flow rate. The visual indicator 13 may be binary or proportional as previously mentioned.

In at least one embodiment, the exhalation indicator 13 may be capable of indicating the decaying exponential phenomena of a breath in which the initial instances of exhalation exhibit higher flow rates than the final moments of exhalation. In some instances, it may be advantageous to determine whether a patient is exhaling throughout the entire time period of exhalation to determine, for instance, whether a patient has completed exhalation. The exhalation indicator 13 may be sensitive to low gas flow velocities and volumes such that it provides an indication of exhalation as long as exhalation is occurring, i.e., as long as gas is still being exhaled from the lungs, even at the very low flow rates typical of end exhalation. A user may be instructed to not force gases into a patient as long as the exhalation indicator indicates exhalation is occurring, thereby preventing breath stacking.

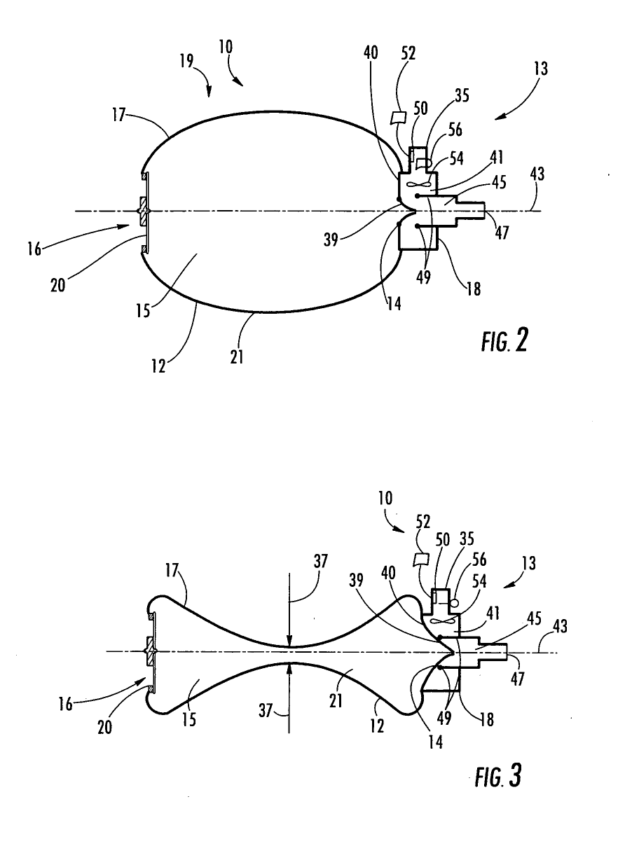

The self-inflating resuscitation system 10 may also include one or more sensors 50 for determining whether a patient is exhaling, as shown in FIGS. 2-4 and 6-10. Upon determining that the patient has exhaled, the sensor 50 may send a signal to a controller 52, processor or other appropriate device. The controller 52 may activate one or more exhalation indicators 13 to notify a caregiver that the patient has either exhaled or not, even though the self-inflating resuscitation bag 12 has inflated. The sensor 50 may be any sensor or combination of sensors capable of sensing whether a patient has exhaled or not. For example, the sensor 50 may be a carbon dioxide sensor, a humidity sensor, a temperature sensor, a flowmeter, an anemometer, or other appropriate device. Any of these sensors 50 may be configured such that a reading below or above a particular threshold causes the sensor to either activate or deactivate the exhalation indicator 13. The threshold may be established based upon data corresponding to breathing patients and data corresponding to non-breathing patients.

A shown in FIG. 1, the self-inflating resuscitation system 10 may include a self-inflating resuscitation bag 12. The self-inflating resuscitation bag 12 may be formed from a generally elongated, flexible squeeze chamber 15. The chamber 15 may be formed from at least one flexible outer wall 17 having a resting position 19, as shown in FIGS. 1 and 2, at which the chamber 15 may be expanded to form a cavity holding a gas and may have at least a minimal amount of structural integrity such that when a compressive force is removed from an outer surface 21 of the outer wall 17, the outer wall 17 returns to the resting position 19. The self-inflating resuscitation bag 12 may be formed of a transparent or translucent plastic and may be readily deformed with hand pressure applying a compressive force to the outer wall 17. The self-inflating resuscitation bag 12 may be formed from other appropriate materials. The self-inflating resuscitation bag 12 may include an outlet port 14 at a first end and intake port 16 at a second end. An outlet valve assembly 18 may be coupled to the self-inflating resuscitation bag 12 adjacent to the outlet port 14. An inlet valve 20 may be coupled to the intake port 16, which may be positioned upstream from the inlet valve 20. The inlet valve 20 may be any valve capable of permitting gases to flow through the valve 20 and into the chamber 15 while preventing gases from flowing out the chamber 15 through the intake port 16. The self-inflating resuscitation system 10 may include an exhalation outlet port 35 extending from the outlet valve assembly 18.

As shown in FIG. 1, an airway device 22 may extend from the outlet valve assembly 18 and be configured to be coupled to an airway of a patient. The airway device 22 may be an endotracheal tube 23, a facemask 24, a supra-laryngeal mask, a conduit 29, a connector 68, a laryngeal mask airway, a COPA tube, a combitube or other appropriate device. The airway device 22, such as the conduit 29, may extend from the outlet valve assembly 18 and may be coupled to a facemask 24. The airway device 22 may be used to place the self-inflating resuscitation bag 12 in communication with the airway of a patient enabling gases to flow from the self-inflating resuscitation bag 12 and into the patient. The facemask 24 may be any conventional facemask capable of providing a seal at a mouth and nose region of a patient.

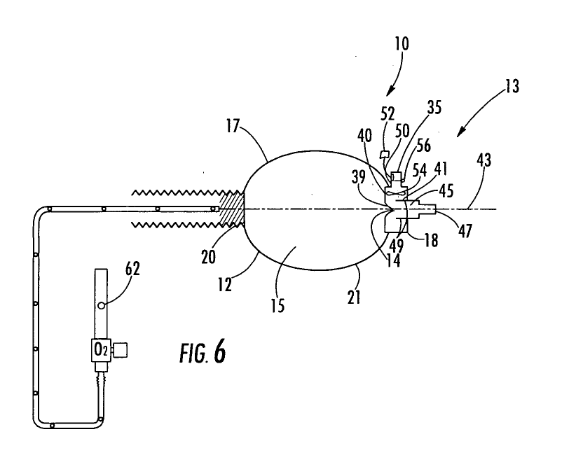

As shown in FIG. 1, the self-inflating resuscitation system 10 may include an entrainment reservoir 33 in communication with the intake port 16. The entrainment reservoir 33 may be configured to attach the self-inflating resuscitation bag 12 to an external gas source (not shown), such as, but not limited to, an enriched oxygen mixture source 62, as shown in FIG. 6, medication sources, and other appropriate sources. The entrainment reservoir 33 may be formed from a flexible and extensible corrugated conduit 26 and tubing 28. Alternately, the entrainment reservoir 33 may be formed from a flaccid collapsible bag (not shown). The tubing 25 delivers gas to entrainment reservoir 33 and may be joined to an external gas source to regulate the type of gas being supplied to the self-inflating resuscitation bag 12. In this manner, gas composition to a patient may be more accurately controlled.

As shown in FIGS. 2-4 and 6-10, the self-inflating resuscitation system 10 may include a sensor 50. The sensor 50 may be positioned in the outlet valve assembly 18. In at least one embodiment, as shown in FIG. 2, the sensor 50 may be positioned in close proximity to the exhalation outlet port 35 to sense the selected parameter or parameters for determining whether the patient has exhaled. The parameters that may be measured may include but are not limited to, carbon dioxide, humidity, temperature, gas velocity, volume, flow rate, or other appropriate parameters. The sensor 50 may be in communication with a controller 52. The controller 52 may be any programmable controller, such as, but not limited to a microcontroller, computer, or other appropriate device for controlling operation of the sensor 50.

The sensor 50 may function by sensing a parameter and then sending a signal to the controller 52. The controller 52 may compare the reading to a predetermined threshold established for an average person exhaling or to any other level selected by the caregiver. If the sensed reading meets or exceeds the parameter or parameters, the controller 52 may activate or deactivate the exhalation indicator 13. In at least one embodiment, activation or deactivation of the exhalation indicator 13 may provide an audible or visual indication, or both, to a caregiver that the patient exhaled. If the reading does not meet or exceed the parameter, then the exhalation indicator 13 is not activated or deactivated, thereby indicating to the caregiver that the patient did not exhale. In one embodiment, the controller 52 may be configured to generate an alarm to indicate inadequate exhalation in situations where a patient has exhaled but has not exhaled an amount sufficient to meet a predetermined threshold. The alarm may be a visual or audible alarm, or both, and may be distinguishable from the exhalation indicator 13.

In yet another embodiment, the self-inflating resuscitation system 10 may include a exhalation indicator 13 in communication with an airway device 22. The airway device 22 may be coupled to a patient to deliver gases, such as, but not limited to, air, oxygen, and other appropriate gases, to the patient. The exhalation indicator 13 is not required to be coupled to a self-inflating resuscitation bag 12. Rather, other positive pressure ventilation sources may be used to supply gases to a patient. The positive pressure ventilation sources may be any source of gases capable of safely delivering gases to a patient.

The self-inflating resuscitation system 10 may be usable to deliver gas into an airway of a patient to facilitate breathing. FIGS. 2 and 3 depict an embodiment of the self-inflating resuscitation system 10 during inspiration. FIG. 2 depicts the self-inflating resuscitation bag 12 in a fully inflated, resting position 19. FIG. 3 depicts the self-inflating resuscitation bag 12 undergoing a compressive force 37. The self-inflating resuscitation bag 12 may include an inlet valve 20 configured to seal intake port 16 during inspiration, thereby forcing the gas contained in the self-inflating resuscitation bag 12 to be directed through the outlet port 14 and into the outlet valve assembly 18 when the self-inflating resuscitation bag 12 is subjected to a compressive force 37. The compressive force 37 may be created by a caregiver's hand or other means.

As shown in FIG. 2, the outlet valve assembly 18 may be formed from an outlet valve 39. In one embodiment, the outlet valve 39 may be coupled to the outlet port 14 of the self-inflating resuscitation bag 12. The outlet valve 39 may be configured such that gases may be capable of flowing out of the self-inflating resuscitation bag 12 through the outlet valve 39; however, gases may be prevented from flowing into the self-inflating resuscitation bag 12 through the outlet valve 39. The outlet valve 39 may be formed from any valve configured to accomplish this objective.

The outlet valve assembly 18 may also include a first chamber 41. The first chamber 41 may extend generally orthogonal to a longitudinal axis 43 of the outlet valve assembly 18. The exhalation outlet port 35 may be positioned in the first chamber 41 at an end of the chamber 41. The outlet valve assembly 18 may also include a second chamber 45 extending from the first chamber 41. In at least one embodiment, the second chamber 45 may be positioned generally along the longitudinal axis 43 of the outlet valve assembly 18 and may be positioned generally orthogonal to the first chamber 41. The outlet valve assembly 18 may include outlet 47 that is configured to be attached to an airway device 22 for delivering gas to and receiving gas from a patient.

During inspiration, gas from the self-inflating resuscitation bag 12 flows through outlet valve 39, into the outlet valve assembly 18 and is delivered to the patient through the outlet 47. As shown in FIG. 3, the outlet valve 39 may be positioned within the outlet valve assembly 18 such that when the outlet valve 39 opens to permit gases to flow out of the self-inflating resuscitation bag 12, the outlet valve 39 at least substantially seals the exhalation outlet port 35 in the first chamber 41. In particular, as shown in FIG. 3, the outlet valve 39 contacts outer walls 49 defining the second chamber 45, thereby sealing the exhalation outlet port 35. A sidewall 40 of the self-inflating resuscitation bag 12 may be flexible to an extent that as gas is forced from the self-inflating resuscitation bag 12, the exhalation outlet port 35 is sealed.

During exhalation, the outlet valve 39 is closed to prevent gases from entering the self-inflating resuscitation bag 12 and thus, opens the exhalation outlet port 35. The resuscitation bag 12 may self-inflate from the position shown in FIG. 3 to the position shown in FIG. 2 by removing the compressive force 37 from the outer wall 17 of the self-inflating resuscitation bag 12. Gases may be drawn into the self-inflating resuscitation bag 12 through the inlet valve 20 at the intake port 16.

As shown in FIGS. 2-4 and 6-10, the self-inflating resuscitation system 10 may include one or more exhalation indicators 13. In at least one embodiment, the exhalation indicators 13 may be positioned within the first chamber 41 between the exhalation outlet port 35 and the second chamber 45. The first chamber 41 may include one or more exhalation indicators 13. In one embodiment, only a single exhalation indicator 13 may be positioned within the first chamber 41. In another embodiment, a plurality of exhalation indicators may be positioned with the first chamber 41. For instance, as shown in FIG. 2, a first exhalation indicator 54 may be a visual indicator, such as any of the visual indicators previously set forth, and a second exhalation indicator 56 may be an audible indicator, such as any of the audible indicators previously set forth. In one embodiment, as shown in FIG. 2, the first exhalation indicator 54 may be a turbine, and the second exhalation indicator 56 may be a whistle.

In another embodiment, as shown in FIG. 4, the exhalation indicators 54, 56 may be positioned downstream of the exhalation outlet port 35. Positioning the exhalation indicators 54, 56 downstream of the exhalation port 35 may increase the ability of the exhalation indicators 54, 56 to detect exhalation of a patient at low flow rates, such as at end portions of exhalation.

Exhalation by a patient forces gases through the airway device 22, through the second chamber 45, and into the first chamber 41. The gases pass the exhalation indicators 54, 56 in the first chamber 41. The flowing gases causes the exhalation indicators 54, 56 to create an audible and visual indication that a patient has exhaled. In at least one embodiment, the exhalation indicators 54, 56 do not indicate that a patient has exhaled until exhalation has reached a predetermined threshold correlating with what defines a predetermined adequate breathing pattern for that patient. The threshold may be determined based upon factors, such as, but not limited to, whether the patient is an adult, child, male, or female, or other appropriate factors, such as body mass. Therefore, the person operating the self-inflating resuscitation bag 12 may become aware that the patient was not exhaling, even though the self-inflating resuscitation bag 12 inflated with ambient air or an O₂-enriched gas mixture.

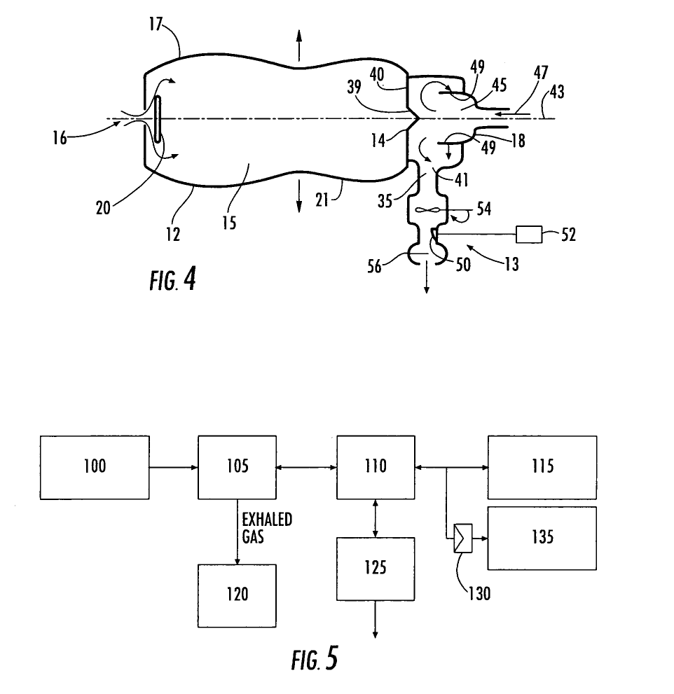

FIG. 5 depicts a schematic representation of gas flow of an embodiment of the self-inflating resuscitation system 10. In this embodiment, a positive pressure ventilation apparatus 100 may be connected via a valve assembly 105 to an airway device 110 to deliver gas to the lungs 115 of a patient. The exhalation port of the valve assembly 105 is connected to an indicator module 120 that includes at least one exhalation indicator for determining the presence and optionally the magnitude of exhaled gas and, therefore, whether the patient is exhaling. The airway device 110 may not provide a perfect seal (as in the case of a face mask) or may have a leak (as in the case of an underinflated endotracheal tube cuff). Thus, a portion of the tidal volume, which may be inspired or exhaled, or both, may be diverted directly to the atmosphere during both inspiration and expiration. The diverted volume is thus not available to activate the indicators in module 120 and thus a poor seal or a leak may be detected. Module 125 represents a leak or poor seal in the airway device. The gastric sphincter is represented as a check valve 130 that tends to trap gas within the stomach 135. In the case of an unprotected airway, gas administered to a patient under positive pressure may flow to both the lungs 115 and the stomach 135. However, the gastric sphincter 130 tends to trap the gas delivered to the stomach and thus, patient exhalation may be reduced in the event of gastric trapping. Nonetheless, the self-inflating resuscitation system 10 may be used to identify this event. In the case of esophageal intubation, gastric sphincter 130 may also trap gas. Such a condition may also be identified using the self-inflating resuscitation system 10.

In an alternative embodiment, the self-inflating resuscitation system 10 may be configured such that if the sensed reading or readings of the exhalation indicators 13 met or exceeded the parameter or parameters, then the one or more exhalation indicators 13 would not be activated and only if the readings did not meet or exceed the parameter or parameters, then would the one or more indicators be activated. In this embodiment, the activation of the one or more exhalation indicators 13 acts as a warning to a caregiver that a patient is not exhaling as intended.

In yet another alternative embodiment, the self-inflating resuscitation system 10 may be configured such that an exhalation indicator 13 may determine whether a breath has been delivered too quickly. If a breath is delivered too quickly and forcefully to an unprotected airway, such as during SIRB ventilation with a face mask, a significant portion of the breath may go the stomach instead of the lungs. Such user errors have led to recent CPR recommendations for delivering a breath slowly to reduce gastric insufflation.

The self-inflating resuscitation system 10 may also be used to detect reduced exhalation as a result of gastric trapping which in turn results from poor inflation technique during positive pressure ventilation of an unprotected airway. The exhalation indicators 13 may be of different sensitivities. For instance, the exhalation indicators 13 may be configured to detect a breath of 50 ml, 100, ml, 200 ml, or other volumes or flow rates of different magnitudes. The exhalation indicators 13 of varying sensitivity may be used simultaneously or may be used individually with the sensitivity matched to the application. The exhalation indicators 13 may be removably attached.

In at least one embodiment, the self-inflating resuscitation system 10 may be formed from a kit configured to be adapted to conventional resuscitation systems to improve the quality of care capable of being administered using conventional systems. The self-inflating resuscitation system 10 may include the outlet valve assembly 18 and one or more exhalation indicators 13 for indicating exhalation of a patient. The outlet valve assembly 18 may be coupled to a conventional system to improve the conventional system. The kit may include any of the previously mentioned components of the self-inflating resuscitation system 10. A resuscitation kit comprising the exhalation indicator 13 can also be used for training novices and practitioners.

A working model of the self-inflating resuscitation system 10 used to evaluate the effectiveness of the system 10 revealed a certain amount of gas trapping due to the flow resistance of the exhalation indicator added to the exhalation port. The gas trapping may be beneficial because the trapped gas provides a certain level of positive end-expiratory pressure (PEEP) that helps maintain the alveoli open. However, should the gas trapping be determined to be excessive, the PEEP may be reduced by using exhalation indicators with features that promote lower flow resistance such as wider flow passages and audible indicators with lower frequencies that require less energy to activate. The self-inflating resuscitation system 10 may also include by-pass flow passages that circumvent the exhalation indicators to prevent the exhalation indicators from restricting flow of gases during exhalation. The self-inflating resuscitation system 10 may also include pressure-threshold devices that open to relieve pressure when a gas pressure in the self-inflating resuscitation system 10 exceeds a predetermined value to reduce gas trapping. The pressure-threshold devices may be, but are not limited to spring loaded pressure relief valves and other appropriate devices.

A working model of the self-inflating resuscitation system 10 was evaluated on a patient simulator where an airway obstruction was deliberately created. Upon squeezing the self-inflating resuscitation bag 12, all the gas escaped via the airway device 22 (in this case a facemask), even though one person was using two hands to seal the facemask and the other was squeezing the bag. The exhalation indicator 18 did not sound upon exhalation, correctly indicating the airway obstruction.

Additionally, a working model of the self-inflating resuscitation system 10, implemented by adding a reed whistle as an audible indicator to the exhalation port of a self-inflating resuscitation system 10 was evaluated by participants in ACLS (Advanced Cardiac Life Support) courses. In randomized order, each participant provided two sets of breaths (with and without audible feedback) to a Human Patient Simulator, modified to log lung volume. Delivered tidal volume (VT) was calculated from the resulting volume trace. The last three breaths in each set were used to compare average VT under both conditions. Eighty seven participants (54 males, 33 females) with clinical training averaging 6.4±9.4 years took part in the study. Average VT delivered with the standard SIRB was 486±166 ml and 624±96 ml with the SIRB incorporating an audible exhalation indicator. Average VT delivered while using an audible indicator of exhalation was 40 percent greater when it followed standard self-inflating resuscitation bag use and 19 percent greater when using the self-inflating resuscitation bag 12 with audible feedback first. The study indicated that use of a self-inflating resuscitation bag 12 with an audible indicator of exhalation improved mask ventilation of a patient simulator suggesting that mask ventilation of a patient with a self-inflating resuscitation bag may also be improved by an objective, real-time feedback of exhaled VT.

Additionally, the self-inflating resuscitation system 10 may be configured so that the self-inflating resuscitation bag 12 may be used as an esophageal intubation detector. The intake port 16 may be configured to be a standard connector, such as a 15 mm diameter connector, that mates to the proximal connector of an endotracheal tube 23. In a situation where a patient is already intubated and where the placement of the tube needs to be confirmed, the self-inflating resuscitation bag 12 may be squeezed and held collapsed. The intake port 16 may be connected to the proximal end of the endotracheal tube 23, and the self-inflating resuscitation bag 12 may inflate by removing compressive pressure from the self-inflating resuscitation bag 12, such as by stopping squeezing the self-inflating resuscitation bag 12. If the self-inflating resuscitation bag 12 inflates upon letting go, the tube 23 is in the trachea because gas in the lungs flows into the self-inflating resuscitation bag 12 to fill it. If the tube 23 is in the esophagus, the self-inflating resuscitation bag 12 stays collapsed or fills slowly because the gastric sphincter limits gas flow from flowing out of the esophagus.

The self-inflating resuscitation system 10 may also be used as an emergency suctioning device, such as in situations where suction devices or a vacuum source are not readily available. The self-inflating resuscitation bag 12 may be configured so that the intake port 16 that is in connection with the self-inflating resuscitation bag 12 is compatible with tubes and catheters used for suctioning. The intake port 16 may be adapted to be compatible with suction equipment and may be removably attached to the self-inflating resuscitation bag 12. Thus, after using the SIRB to suction and collect the aspirate in the bag, the intake port 16 and the inlet valve 20 may be removed to empty the self-inflating resuscitation bag 12 to facilitate subsequent use either as a suctioning device or as a ventilation device. The intake port 16 and the inlet valve 20 may together form an inlet assembly that may be easily removable from the self-inflating resuscitation bag 12. In at least one embodiment, the inlet assembly may be screwed onto the self-inflating resuscitation bag 12 via a large threaded port.

The foregoing is provided for purposes of illustrating, explaining, and describing embodiments of this invention. Modifications and adaptations to these embodiments will be apparent to those skilled in the art and may be made without departing from the scope or spirit of this invention.

We claim:

1. A self-inflating resuscitation system, comprising:
a self-inflating resuscitation chamber formed from at least one flexible outer wall having a resting position at which the self-inflating resuscitation chamber is expanded to form a cavity holding a gas and having sufficient structural integrity such that when a compressive force is removed from an outer surface of the outer wall, the outer wall returns to the resting position;
at least one exhalation indicator coupled to the self-inflating resuscitation chamber to indicate whether a patient to which the self-inflating resuscitation chamber is attached is exhaling.

2. The self-inflating resuscitation system of claim 1, wherein the at least one exhalation indicator is selected from a group consisting of an audible indicator and a visual indicator.

3. The self-inflating resuscitation system of claim 2, wherein the at least one exhalation indicator is an audible indicator selected from a group consisting of a whistle, a reed, a buzzer, a bell, a beeper, and a ringer.

4. The self-inflating resuscitation system of claim 2, wherein the at least one exhalation indicator is a visual indicator selected from a group consisting of at least one light, at least one light-emitting diode, a liquid crystal display, a turbine, and a pneumatic toggled lens.

5. The self-inflating resuscitation system of claim 1, wherein the at least one exhalation indicator comprises an audible indicator and a visual indicator.

6. The self-inflating resuscitation system of claim 5, wherein the at least one exhalation indicator is an audible indicator selected from a group consisting of a whistle, a reed, a buzzer, a bell, a beeper, and a ringer.

7. The self-inflating resuscitation system of claim 5, wherein the at least one exhalation indicator is a visual indicator selected from a group consisting of at least one light, at least one light-emitting diode, a liquid crystal display, a turbine, and a pneumatic toggled lens.

8. The self-inflating resuscitation system of claim 1, wherein the self-inflating resuscitation chamber further comprises an outlet port and the at least one exhalation indicator is positioned in a chamber downstream of the outlet port.

9. A self-inflating resuscitation system, comprising:
a self-inflating resuscitation chamber formed from at least one flexible outer wall having a resting position at which the self-inflating resuscitation chamber is expanded to form a cavity holding a gas and having sufficient structural integrity such that when a compressive force is removed from an outer surface of the outer wall, the outer wall returns to the resting position;
an intake port in the self-inflating resuscitation chamber;
an inlet valve coupled to the intake port allowing gases to flow into the self-inflating resuscitation chamber but restricting gases from flowing out of the self-inflating resuscitation chamber;
an outlet port in the self-inflating resuscitation chamber;
an outlet valve assembly coupled to the outlet port allowing gases to flow out of the self-inflating resuscitation chamber but restricting gases from flowing into the self-inflating resuscitation chamber; and
at least one exhalation indicator coupled to the outlet valve assembly to indicate whether a patient to which the self-inflating resuscitation chamber is attached is exhaling.

10. The self-inflating resuscitation system of claim 9, wherein the outlet valve assembly comprises an outlet valve coupled to the outlet port, a first chamber having an exhalation outlet port, and a second chamber extending from the first chamber and having a airway device.

11. The self-inflating resuscitation system of claim 8, wherein the airway device comprises a facemask configured to cover a mouth and nose region of a patient.

12. The self-inflating resuscitation system of claim 8, wherein the airway device comprises a connector configured to be coupled to as device selected from the group consisting of an endotracheal tube, a laryngeal mask airway, a supra laryngeal mask airway, a COPA tube, and a combitube.

13. The self-inflating resuscitation system of claim 10, wherein the at least one exhalation indicator is positioned in the first chamber of the outlet valve assembly.

14. The self-inflating resuscitation system of claim 10, wherein the outlet valve is positioned such that when the outlet valve opens to permit gases to flow out of the self-inflating resuscitation chamber, the outlet valve at least substantially seals the exhalation outlet port in the first chamber.

15. The self-inflating resuscitation system of claim 9, further comprising an oxygen source coupled to the intake port in the self-inflating resuscitation chamber.

16. The self-inflating resuscitation system of claim 9, wherein the at least one exhalation indicator is selected from a group consisting of an audible indicator and a visual indicator.

17. The self-inflating resuscitation system of claim 16, wherein the at least one exhalation indicator is an audible indicator selected from a group consisting of a whistle, a reed, a buzzer, a bell, a beeper, and a ringer.

18. The self-inflating resuscitation system of claim 16, wherein the at least one exhalation indicator is a visual indicator selected from a group consisting of at least one light, at least one light-emitting diode, a liquid crystal display, a turbine, and a pneumatic toggled lens.

19. The self-inflating resuscitation system of claim 9, wherein the at least one exhalation indicator comprises an audible indicator and a visual indicator.

20. The self-inflating resuscitation system of claim 19, wherein the at least one exhalation indicator is an audible indicator selected from a group consisting of a whistle, a reed, a buzzer, a bell, a beeper, and a ringer.

21. The self-inflating resuscitation system of claim 19, wherein the at least one exhalation indicator is a visual indicator selected from a group consisting of at least one light, at least one light-emitting diode, a liquid crystal display, a turbine, and a pneumatic toggled lens.

22. The self-inflating resuscitation system of claim 19, further comprising a sensor for sensing at least one parameter and a controller coupled to the sensor for receiving signals from the sensor and for controlling activation of the at least one exhalation indicator.

23. The self-inflating resuscitation system of claim 9, wherein the at least one exhalation indicator is positioned downstream of the outlet port.

24. A method of determining whether a patient is adequately ventilated, comprising:
attaching a self-inflating resuscitation chamber to the patient, wherein the self-inflating resuscitation chamber comprises at least one flexible outer wall having a resting position at which the self-inflating resuscitation chamber is expanded to form a cavity holding a gas and having sufficient structural integrity such that when a compressive force is removed from an outer surface of the outer wall, the outer wall returns to the resting position and including at least one exhalation indicator coupled to the self-inflating resuscitation chamber to indicate whether a patient to which the self-inflating resuscitation chamber is attached is exhaling;
applying a compressive force to the self-inflating resuscitation chamber to expel the gas from the self-inflating resuscitation chamber into the patient; and
monitoring the at least one exhalation indicator to determine whether the patient exhales.

25. The method of claim 24, wherein attaching a self-inflating resuscitation chamber on the patient comprises attaching a self-inflating resuscitation chamber on the patient, wherein the at least one exhalation indicator is selected from a group consisting of an audible indicator and a visual indicator.

26. The method of claim 25, wherein attaching a self-inflating resuscitation chamber on the patient comprises attaching a self-inflating resuscitation chamber on the patient, wherein the at least one exhalation indicator is an audible indicator selected from a group consisting of a whistle, a reed, a buzzer, a bell, a beeper, and a ringer.

27. The method of claim 25, wherein attaching a self-inflating resuscitation chamber on the patient comprises attaching a self-inflating resuscitation chamber on the patient, wherein the at least one exhalation indicator is a visual indicator selected from a group consisting of least one light, at least one light-emitting diode, a liquid crystal display, a turbine, and a pneumatic toggled lens.

28. The method of claim 24, wherein attaching a self-inflating resuscitation chamber on the patient comprises attaching a self-inflating resuscitation chamber on the patient, wherein the self-inflating resuscitation chamber further comprises an intake port in the self-inflating resuscitation chamber, an inlet valve coupled to the intake port allowing gases to flow into the self-inflating resuscitation chamber but restricting gases from flowing out of the self-inflating resuscitation chamber, an outlet port in the self-inflating resuscitation chamber, and an outlet valve assembly coupled to the outlet port allowing gases to flow out of the self-inflating resuscitation chamber but restricting gases from flowing into the self-inflating resuscitation chamber.

29. The method of claim 28, wherein attaching a self-inflating resuscitation chamber on the patient comprises attaching a self-inflating resuscitation chamber on the patient, wherein the outlet valve assembly comprises an outlet valve coupled to the outlet port, a first chamber having an exhalation outlet port, and a second chamber extending from the first chamber and having a airway device, wherein the outlet valve is positioned such that when the outlet valve opens to permit gases to flow out of the self-inflating resuscitation chamber, the outlet valve at least substantially seals the exhalation outlet port in the first chamber.

30. A method of determining esophageal intubation in an intubated patient comprising:
applying a compressive force to a self-inflating resuscitation chamber to expel gas from the self-inflating resuscitation chamber;
attaching the self-inflating resuscitation chamber to an endotracheal tube installed in the patient, wherein the self-inflating resuscitation chamber comprises at least one flexible outer wall having a resting position at which the self-inflating resuscitation chamber is expanded to form a cavity holding a gas and having sufficient structural integrity such that when a compressive force is removed from an outer surface of the outer wall, the outer wall returns to the resting position; and
monitoring refilling of the self-inflating resuscitation chamber to determine whether the endotracheal tube is in lungs or stomach of the patient.

31. A self-inflating resuscitation system for human resuscitation, comprising:
an outlet valve assembly adapted to be coupled to an outlet port of a self-inflating resuscitation chamber and including an outlet valve adapted to allow gases to flow out of the self-inflating resuscitation chamber but restricting gases from flowing into the self-inflating resuscitation chamber and adapted to be coupled to an airway device extending from an airway of a patient; and
at least one exhalation indicator coupled to the outlet valve assembly to indicate whether a patient to which the self-inflating resuscitation chamber is attached is exhaling.

32. The self-inflating resuscitation system of claim 31, wherein the outlet valve assembly comprises a first chamber having an exhalation outlet port and a second chamber extending from the first chamber and adapted to be coupled to the airway device.

33. The self-inflating resuscitation system of claim 32, wherein the at least one exhalation indicator is positioned in the first chamber of the outlet valve assembly.

34. The self-inflating resuscitation system of claim 31, wherein the outlet valve is positioned such that when the outlet valve opens to permit gases to flow out of the self-inflating resuscitation chamber, the outlet valve at least substantially seals the exhalation outlet port in the first chamber.

35. The self-inflating resuscitation system of claim 31, wherein the at least one exhalation indicator is selected from a group consisting of an audible indicator and a visual indicator.

36. The self-inflating resuscitation system of claim 35, wherein the at least one exhalation indicator is an audible indicator selected from a group consisting of a whistle, a reed, a buzzer, a bell, a beeper, and a ringer.

37. The self-inflating resuscitation system of claim 35, wherein the at least one exhalation indicator is a visual indicator selected from a group consisting of at least one light, at least one light-emitting diode, a liquid crystal display, a turbine, and a pneumatic toggled lens.

38. A device for determining esophageal intubation in an intubated patient comprising:
a self-inflating resuscitation chamber formed from an inlet port, an outlet port, and at least one flexible outer wall having a resting position at which the self-inflating resuscitation chamber is expanded to form a cavity holding a gas and having sufficient structural integrity such that when a compressive force is removed from an outer surface of the outer wall, the outer wall returns to the resting position; and
an endotracheal tube coupled to the inlet port of the self-inflating resuscitation chamber.

39. A method of determining whether a patient is adequately ventilated, comprising:
attaching an airway device to the patient, wherein the airway device is coupled to a positive pressure ventilation source;
expelling a gas from the positive pressure ventilation source into the patient; and
monitoring at least one exhalation indicator in communication with the airway device to determine whether the patient exhales.
